(19) United States
(12) Patent Application Publication
(10) Pub. No.: US 2018/0352348 A1
(43) Pub. Date: Dec. 6, 2018

(54) BONE CONDUCTION DEVICE

(71) Applicant: Sonitus Technologies Inc., Osaka (JP)

(72) Inventors: Munenori AOYAGI, Kumamoto (JP); Hirohiko OOWAKI, Fukuoka (JP); Shinji TANAKA, Saga (JP); Michael PARE, San Carlos, CA (US); Tim PROULX, Santa Cruz, CA (US)

(73) Assignee: Sonitus Technologies Inc., Osaka (JP)

(21) Appl. No.: 15/615,105

(22) Filed: Jun. 6, 2017

(51) Int. Cl. H04R 25/00

(52) U.S. Cl. CPC H04R 25/606; H04R 25/353; A61H 23/004; A61M 2205/3375; H04R 25/43

(57) ABSTRACT

A bone conduction device includes: a case; a vibrating body disposed in the case; and a first weight held by the vibrating body vibrating body. The vibrating body includes a support portion supported by the case and a first holding portion that holds the first weight. A center of mass of the first weight is located outside a first normal region of the vibrating body in the first holding portion.

### FIELD

The present disclosure relates to a device that causes a sound signal to propagate through an organism such as a bone, and/or a device that extracts, as a sound signal, vibrations that have propagated through an organism.

### BACKGROUND

Methods for recognizing sound include a method using an air-conducted sound, and a method using a bone-conducted sound. The method using an air-conducted sound recognizes sound by detecting, with an eardrum, a sound propagating through the air as a medium. The method using a bone-conducted sound recognizes a sound propagating though an organism such as a skull bone.

A hearing-impaired person and a person under a noise environment may be able to recognize sound via a bone-conducted sound even when they cannot recognize an air-conducted sound.

The sound recognition method using the bone-conducted sound uses a bone conduction device. Bone conduction devices include those having the function of converting an electric signal of sound into a bone-conducted sound, such as a bone conduction speaker, those having the function of converting a bone-conducted sound into an electric signal, such as a bone conduction microphone, and those having both of these functions.

### CITATION LIST

### Patent literature

[Patent Literature 1] Japanese Unexamined Patent Application Publication No. H08-195995

### SUMMARY

### Technical Problem

A bone conduction device is known in which a weight is attached to an end of a cantilever beam (Patent Literature 1). It is generally known that attaching a weight to a beam results in a reduction in the resonance frequency, and the larger the mass of the weight, the greater the reduction in the resonance frequency is.

A bone conduction device is mainly used while attached to a human body, and thus may be reduced in size. Of course, the size of a battery for driving the bone conduction device may also be reduced. The size reduction of the battery leads to a reduced consumable power or a shortened driving time of the bone conduction device. A reduced consumable power leads to a reduction in the output of a bone-conducted sound from the bone conduction device, or a reduction in the output of an electric signal based on a bone-conducted sound.

The present disclosure achieves at least one of a reduced power consumption and an increased output.

### Solution to Problem

The present disclosure is directed to a bone conduction device including: a case that transmits bone-conducted vibrations; a vibrating body disposed in the case; and a first weight held by the vibrating body, wherein the vibrating body includes a support portion supported by the case and a first holding portion that holds the first weight, and a center of mass of the first weight is located outside a first normal region of the vibrating body in the first holding portion.

### Advantageous Effects

A bone conduction device according to the present disclosure achieves at lease one of a reduced power consumption and an increased output.

### BRIEF DESCRIPTION OF DRAWINGS

These and other objects, advantages and features of the disclosure will become apparent from the following description thereof taken in conjunction with the accompanying drawings that illustrate a specific embodiment of the present disclosure.

FIG. 1 is a front cross-sectional view of a bone conduction device according to Embodiment 1 of the present disclosure.

FIG. 2 is a perspective view of a vibrating body, a first weight, and a second weight of the bone conduction device according to Embodiment 1 of the present disclosure.

FIG. 3 is a perspective view of the vibrating body of the bone conduction device according to Embodiment 1 of the present disclosure.

FIG. 4 is a front view of the vibrating body, the first weight, and the second weight of the bone conduction device according to Embodiment 1 of the present disclosure.

FIG. 5 is a graph showing vibration characteristics of the bone conduction device according to Embodiment 1 of the present disclosure and a comparative example.

FIG. 6 is a front cross-sectional view of a bone conduction device according to Variation 1 of Embodiment 1 of the present disclosure.

FIG. 7 is a front cross-sectional view of a bone conduction device according to Variation 2 of Embodiment 1 of the present disclosure.

FIG. 8 is a front cross-sectional view of a bone conduction device according to Variation 3 of Embodiment 1 of the present disclosure.

FIG. 9 is a front cross-sectional view of a bone conduction device according to Variation 4 of Embodiment 1 of the present disclosure.

FIG. 10 is a front cross-sectional view of a bone conduction device according to Variation 5 of Embodiment 1 of the present disclosure.

FIG. 11 is a graph showing vibration characteristics of the bone conduction device according to Variation 5 of Embodiment 1 of the present disclosure and a comparative example.

FIG. 12 is a front cross-sectional view of a bone conduction device according to Variation 6 of Embodiment 1 of the present disclosure.

FIG. 13 is a front cross-sectional view of a bone conduction device according to Variation 7 of Embodiment 1 of the present disclosure.

FIG. 14 is a front cross-sectional view of a bone conduction device according to Embodiment 2 of the present disclosure.

FIG. 15 is a front cross-sectional view of a bone conduction device according to Variation 1 of Embodiment 2 of the present disclosure.

FIG. 16 is a front cross-sectional view of a bone conduction device according to Embodiment 3 of the present disclosure.

FIG. 17 is a top view of the bone conduction device according to Embodiment 3 of the present disclosure.

FIG. 18 is a cross-sectional view taken along the line A-A in FIG. 16.

FIG. 19 is a front cross-sectional view of a bone conduction device according to Variation 1 of Embodiment 3 of the present disclosure.

FIG. 20 is a top view of the bone conduction device according to Variation 1 of Embodiment 3 of the present disclosure.

FIG. 21 is a cross-sectional view taken along the line A-A in FIG. 19.

FIG. 22 is a graph showing vibration characteristics of the bone conduction device according to Embodiment 3 of the present disclosure and Variation 1 thereof, as well as a comparative example.

FIG. 23 is a front cross-sectional view of a bone conduction device according to Variation 2 of Embodiment 3 of the present disclosure.

FIG. 24 is a cross-sectional view taken along the line A-A in FIG. 23.

FIG. 25 is a graph showing vibration characteristics of the bone conduction device according to Variation 2 of Embodiment 3 of the present disclosure and comparative examples.

### DESCRIPTION OF EMBODIMENTS

### Embodiment 1

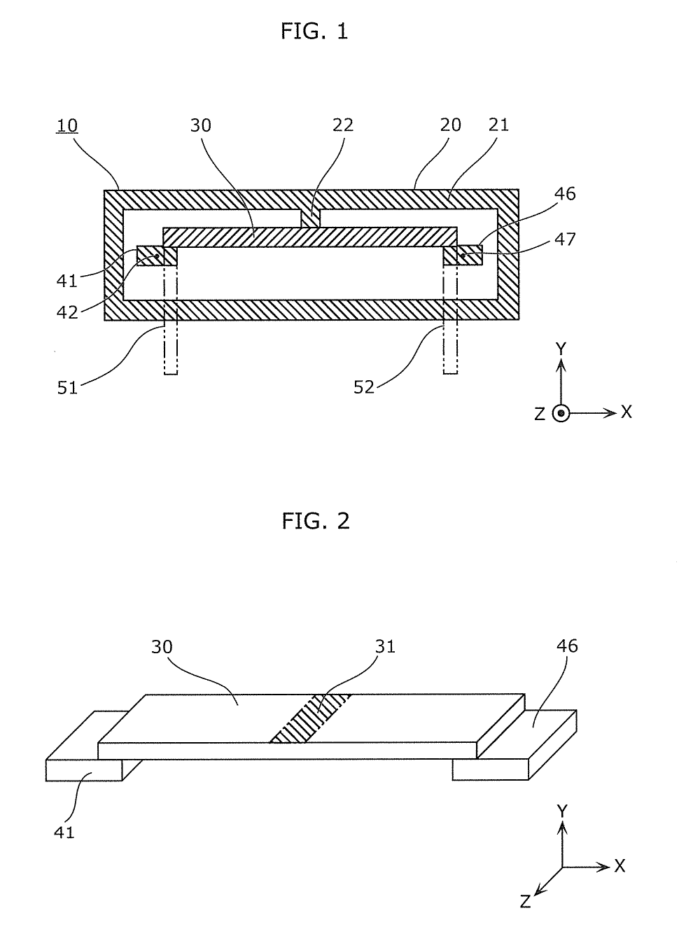

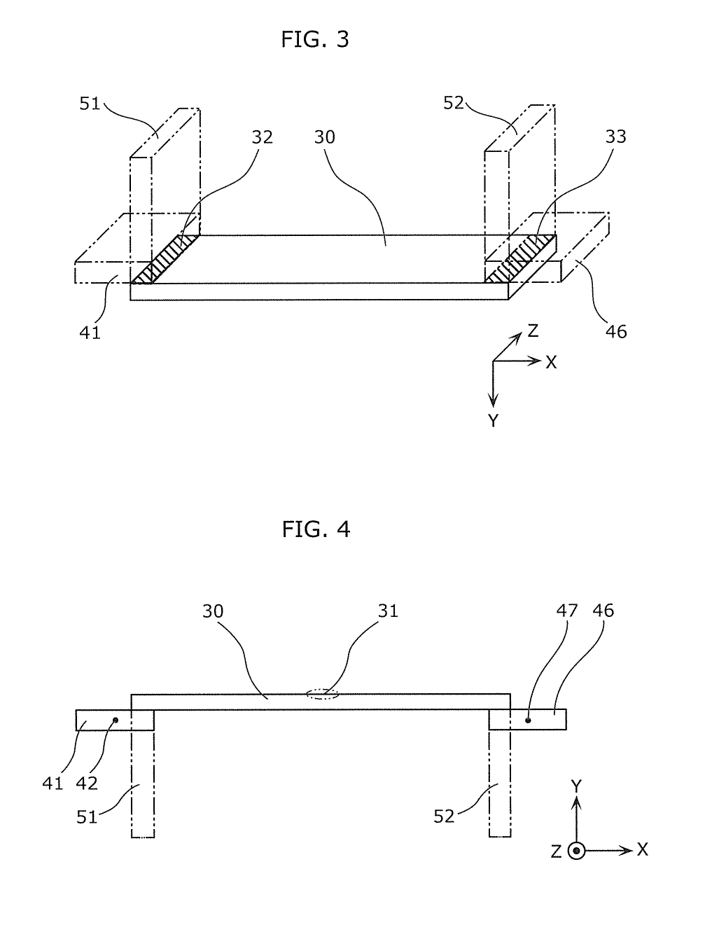

Hereinafter, a bone conduction device according to Embodiment 1 of the present disclosure will be described. FIG. 1 is a front cross-sectional view of a bone conduction device according to Embodiment 1 of the present disclosure, FIG. 2 is a perspective view of a vibrating body, a first weight, and a second weight of the bone conduction device according to Embodiment 1 of the present disclosure, FIG. 3 is a perspective view of the vibrating body of the bone conduction device according to Embodiment 1 of the present disclosure, and FIG. 4 is a front view of the vibrating body, the first weight, and the second weight of the bone conduction device according to Embodiment 1 of the present disclosure. FIG. 2 is a perspective view of the vibrating body, the first weight, and the second weight as viewed from above the plane of the paper in FIG. 1. In FIG. 2, the hatched region indicates a support portion 31 instead of the cross section. FIG. 3 is a perspective view of the vibrating body as viewed from below the plane of the paper in FIG. 2. In FIG. 3, the hatched regions indicate a first holding portion 32 and a second holding portion 33 instead of the cross section. The X-axis is an axis whose positive direction extends rightward in the left-right direction on the plane of the paper in FIG. 1. The Y-axis is an axis whose positive direction extends upward in the up-down direction on the plane of the paper in FIG. 1. The Z-axis is an axis whose positive direction extends frontward in the back-and-front direction on the plane of the paper in FIG. 1. The X-, Y-, and Z-axes are the same in FIGS. 1 to 4.

A bone conduction device 10 according to the present disclosure includes a case 20, a vibrating body 30, and a first weight 41. The bone conduction device 10 further includes a second weight 46. The vibrating body 30, the first weight 41, and the second weight 46 are located inside the case 20.

The case 20 is made of resin, for example. The case 20 has a space thereinside, and the vibrating body 30, the first weight 41, and the second weight 46 are located in the space. The case 20 includes a body 21 and a projection 22. The body 21 covers the vibrating body 30, the first weight 41, and the second weight 46, and the projection 22 is provided inside the body 21. The projection 22 supports the vibrating body 30. The projection 22 is sized such that the vibrating body 30 does not come into contact with the body 21 even when the vibrating body 30 vibrates.

The vibrating body 30 has a plate shape having a longitudinal direction, a lateral direction, and a thickness direction. The longitudinal direction is the X-axis direction, the lateral direction is the Z-axis direction, and the thickness direction is the Y-axis direction. The vibrating body 30 has a configuration in which layers of a piezoelectric material are stacked, for example. The vibrating body 30 includes a support portion 31 and a first holding portion 32. The vibrating body 30 further includes a second holding portion 33. The vibrating body 30 is attached to the case 20 as a result of the support portion 31 being fixed to the projection 22. That is, a portion of the vibrating body 30 that is attached to the case 20 is the support portion 31. The first holding portion 32 is a portion of the vibrating body 30 that fixes the first weight 41. The second holding portion 33 is a portion of the vibrating body 30 that fixes the second weight 46. The vibrating body 30 includes the second holding portion 33 on a side opposite to the first holding portion 32 across the support portion 31.

The first weight 41 serves to lower the resonance frequency of the vibrating body 30. The second weight 46 also serves to lower the resonance frequency of the vibrating body 30. The material of the first weight 41 and the second weight 46 may have a higher density in order to reduce the size for the same mass, but may also be determined taking into consideration the processability, the costs, and the like in a comprehensive manner.

A center of mass 42 of the first weight 41 is located at the center of the first weight 41 if the first weight 41 is uniform. A center of mass 47 of the second weight 46 is also located at the center of second weight 46 if the second weight 46 is uniform.

A first normal region 51 is a normal region of the vibrating body 30 in the first holding portion 32. A normal to the vibrating body 30 is defined with respect to a neutral plane in the vibrating body 30. A neutral plane in the vibrating body 30 is a plane that does not stretch or shrink when the vibrating body 30 is flexed by vibrating. Among the normals to the neutral plane, a set of lines extending through the first holding portion 32 constitutes the first normal region 51. The center of mass 42 of the first weight 41 is present outside the first normal region 51.

A second normal region 52 is a normal region of the vibrating body 30 in the second holding portion 33. The second normal region 52 is also defined in the same manner as the first normal region 51. The center of mass 47 of the second weight 46 is present outside the second normal region 52. Although the direction in which the first normal region 51 and the second normal region 52 are spaced away from the vibrating body 30 is depicted as being finite in FIGS. 1, 3 and 4, the direction is actually infinite. The same applies to the following drawings showing the normal regions.

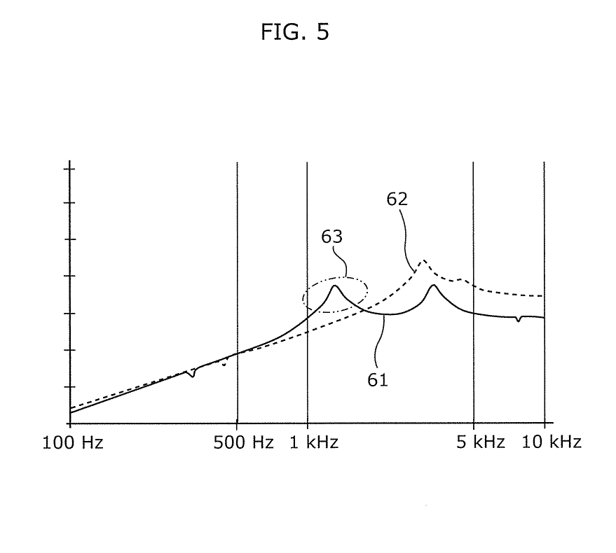

In FIG. 5, the frequency is plotted on the horizontal axis and the output from the vibrating body is plotted on the vertical axis, and the horizontal axis indicates logarithmic coordinates and the vertical axis indicates decibels. The output from the vibrating body is a transfer function between the output voltage determined by measuring the acceleration of the projection 22 by using a laser Doppler vibrometer (LDV) or the like and the input voltage to the piezoelectric material. Vibration characteristics 61 according to the present disclosure represent the vibration characteristics of the bone conduction device 10 shown in FIGS. 1 to 4, and vibration characteristics 62 according to a comparative example represent the vibration characteristics of such a configuration in which the center of mass 42 of the bone conduction device 10 shown in FIGS. 1 to 4 is located inside the first normal region 51, and the center of mass 47 thereof is located inside the second normal region 52.

Whereas the vibration characteristics 62 according to the comparative example have a single output peak, the vibration characteristics 61 according to the present disclosure have, in addition to a peak at substantially the same frequency as that of the output peak of the vibration characteristics 62 according to the comparative example, a low-frequency peak 63 at a frequency lower than that of the peak. Due to the low-frequency peak 63, the bone conduction device 10 according to Embodiment 1 of the present disclosure has a larger output in a frequency band that is slightly higher than 1 kHz. In FIG. 5, a larger output at a certain frequency indicates that the vibrating body 30 is prone to vibrate at that frequency, and it is possible to convert an audio signal corresponding to the frequency into a louder bone-conducted sound, or convert a bone-conducted sound corresponding to the frequency into a larger audio output. Since audio is mainly in a frequency band around 1 kHz, the bone conduction device 10 according to the present disclosure achieves, with a low power consumption, a sufficient output of a sound in a frequency band corresponding to audio.

The reason why the bone conduction device 10 according to the present disclosure has the low-frequency peak 63 also in a lower frequency band as compared with the comparative example seems to be that vibrations different from those in the comparative example occur because the center of mass 42 of the first weight 41 is located outside the first normal region 51, and the center of mass 47 of the second weight 46 is located outside the second normal region 52. That is, the bone conduction device 10 has two vibration systems having different vibration characteristics, so that coupled vibrations, or vibrations similar thereto occur. Specifically, it seems that as a result of the center of mass 42 of the first weight 41 being located outside the first normal region 51, vibrations of the first weight 41 with respect to the first holding portion 32 of the vibrating body 30, and vibrations of the second weight 46 with respect to the second holding portion 33 of the vibrating body 30 exert an influence, aside from vibrations of the vibrating body 30 with respect to the support portion 31. By utilizing the occurrence of a plurality of output peaks of the vibration characteristics as a result of the occurrence of a plurality of vibration systems in this way, it is possible to enhance the vibration characteristics.

By utilizing such vibrations of the first weight 41 and the second weight 46 relative to the vibrating body 30, the bone conduction device 10 according to the present disclosure has the low-frequency peak 63 and enhances the output in an audio frequency band.

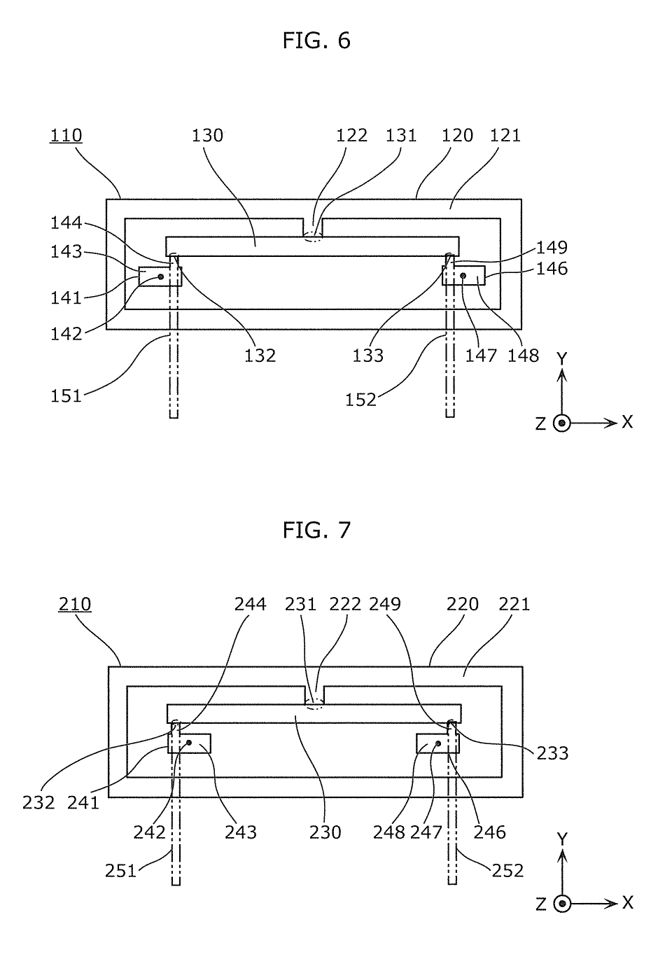

FIG. 6 is a front cross-sectional view of a bone conduction device according to Variation 1 of Embodiment 1 of the present disclosure. In FIG. 6, the hatching indicating a cross section has been omitted to facilitate viewing. The same applies to the following drawings.

A bone conduction device 110, a case 120, a body 121, a projection 122, a vibrating body 130, a support portion 131, a first holding portion 132, a second holding portion 133, a first weight 141, a center of mass 142, a second weight 146, a center of mass 147, a first normal region 151, and a second normal region 152 that are disclosed in FIG. 6 respectively correspond to the bone conduction device 10, the case 20, the body 21, the projection 22, the vibrating body 30, the support portion 31, the first holding portion 32, the second holding portion 33, the first weight 41, the center of mass 42, the second weight 46, the center of mass 47, the first normal region 51, and the second normal region 52 described in FIGS. 1 to 4. The bone conduction device 110 disclosed in FIG. 6 differs from the bone conduction device 10 described in FIGS. 1 to 4 in that the specific shapes of the first weight 141 and the second weight 146 are different from those of the first weight 41 and the second weight 46.

The first weight 141 includes a first weight body 143 and a first connecting portion 144. The first weight body 143 and the first connecting portion 144 constitute an integrated structure, and the first connecting portion 144 is fixed to the first holding portion 132 of the vibrating body 130. A center of mass 142 of the first weight 141 is located outside the first normal region 151. The second weight 146 includes a second weight body 148 and a second connecting portion 149. The second weight body 148 and the second connecting portion 149 constitute an integrated structure, and the second connecting portion 149 is fixed to the second holding portion 133 of the vibrating body 130. A center of mass 147 of the second weight 146 is located outside the second normal region 152.

In the bone conduction device 110 according to Variation 1 of Embodiment 1 of the present disclosure, the center of mass 142 of the first weight 141 is located outside the first normal region 151, and the center of mass 147 of the second weight 146 is located outside the second normal region 152. Accordingly, vibrations of the first weight 141 and vibrations of the second weight 146 relative to the vibrating body 130 occur, so that the bone conduction device 110 achieves, with a low power consumption, a sufficient output of a sound in a frequency band corresponding to audio, based on the same principle as that of the bone conduction device 10 disclosed in FIGS. 1 to 4.

Although the first connecting portion 144 and the first weight body 143 constitute an integrated structure, they may be separate objects. Likewise, the second connecting portion 149 may be an object separate from the second weight body 148.

FIG. 7 is a front cross-sectional view of a bone conduction device according to Variation 2 of Embodiment 1 of the present disclosure. A bone conduction device 210, a case 220, a body 221, a projection 222, a vibrating body 230, a support portion 231, a first holding portion 232, a second holding portion 233, a first weight 241, a center of mass 242, a first weight body 243, a first connecting portion 244, a second weight 246, a center of mass 247, a second weight body 248, a second connecting portion 249, a first normal region 251, and a second normal region 252 according to Variation 2 of Embodiment 1 shown in FIG. 7 respectively correspond to the bone conduction device 110, the case 120, the body 121, the projection 122, the vibrating body 130, the support portion 131, the first holding portion 132, the second holding portion 133, the first weight 141, the center of mass 142, the first weight body 143, the first connecting portion 144, the second weight 146, the center of mass 147, the second weight body 148, the second connecting portion 149, the first normal region 151, and the second normal region 152 according to Variation 1 of Embodiment 1 shown in FIG. 6.

The bone conduction device 210 disclosed in FIG. 7 differs from the bone conduction device 110 disclosed in FIG. 6 in that the orientations of the first weight 241 and the second weight 246 are different from those of the first weight 141 and the second weight 146. Specifically, in the bone conduction device 110 disclosed in FIG. 6, the center of mass 142 of the first weight 141 is at a position further away than the first holding portion 132, and the center of mass 147 of the second weight 146 is at a position further away than the second holding portion 133, with respect to the support portion 131. In contrast, the center of mass 242 of the first weight 241 and the center of mass 247 of the second weight 246 in the bone conduction device 210 disclosed in FIG. 7 are located closer than the first holding portion 232 and the second holding portion 233, respectively, with respect to the support portion 231.

In the bone conduction device 210 disclosed in FIG. 7, the center of mass 242 of the first weight 241 is located outside the first normal region 251, and the center of mass 247 of the second weight 246 is located outside the second normal region 252.

In the bone conduction device 210 disclosed in FIG. 7 as well, vibrations of the first weight 241 and vibrations of the second weight 246 relative to the vibrating body 230 occur, so that the bone conduction device 210 achieves, with a low power consumption, a sufficient output of a sound in a frequency band corresponding to audio, based on the same principle as that of the bone conduction device 10 disclosed in FIGS. 1 to 4.

Since the center of mass 142 of the first weight 141 and the center of mass 147 of the second weight 146 are located at positions further away from the support portion 131, the bone conduction device 110 disclosed in FIG. 6 is more advantageous than the bone conduction device 210 disclosed in FIG. 7 in enhancing the output in a lower frequency band.

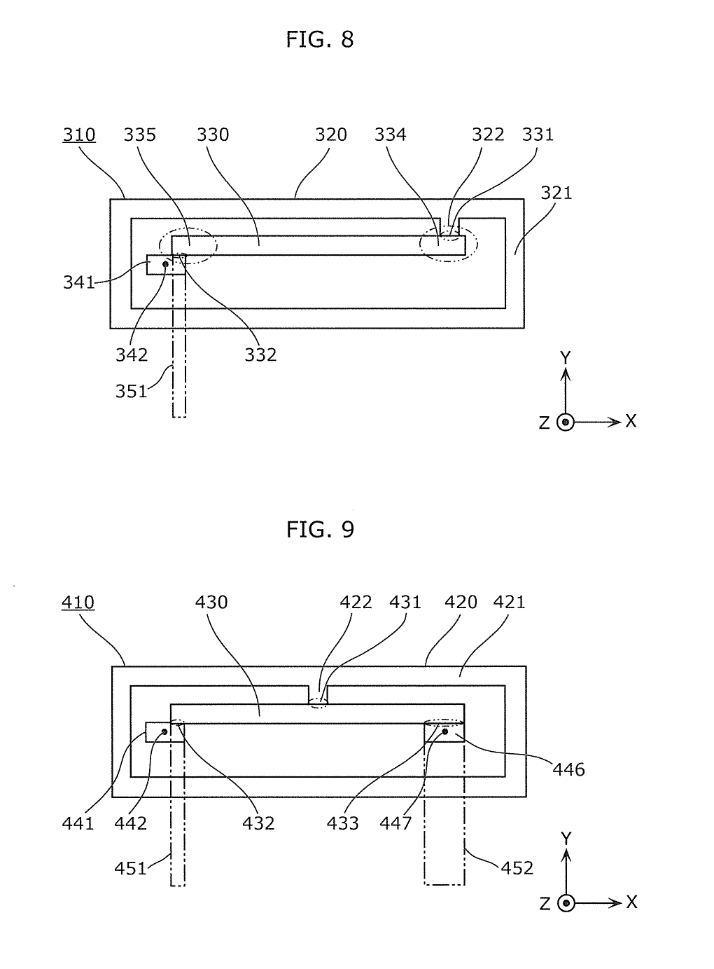

FIG. 8 is a front cross-sectional view of a bone conduction device according to Variation 3 of Embodiment 1 of the present disclosure. A bone conduction device 310, a case 320, a body 321, a projection 322, a vibrating body 330, a support portion 331, a first holding portion 332, a first weight 341, a center of mass 342, and a first normal region 351 that are disclosed in FIG. 8 respectively correspond to the bone conduction device 10, the case 20, the body 21, the projection 22, the vibrating body 30, the support portion 31, the first holding portion 32, the first weight 41, the center of mass 42, and the first normal region 51 that are disclosed in FIGS. 1 to 4. The bone conduction device 310 disclosed in FIG. 8 does not include the second weight 46 of the bone conduction device 10 disclosed in FIGS. 1 to 4. Accordingly, the bone conduction device 310 disclosed in FIG. 8 does not also include constituting elements corresponding to the second holding portion 33, the center of mass 47, and the second normal region 52. The vibrating body 330 includes a first end portion 334 at an end portion in its longitudinal direction (the left-right direction on the plane of the paper in FIG. 8), and a second end portion 335 at an end portion on a side opposite thereto. The first end portion 334 is a region surrounded by the dashed double-dotted line at the right end portion of the vibrating body 330 shown in FIG. 8. The second end portion 335 is a region surrounded by the dashed double-dotted line at the left end portion of the vibrating body 330 shown in FIG. 8. The vibrating body 330 includes the support portion 331 at the first end portion 334, and includes the first holding portion 332 at the second end portion 335.

Since the bone conduction device 310 disclosed in FIG. 8 does not include the second weight 46, the vibrating body 330 can be made longer if the case 320 and the case 20 have the same size. In general, the output in a low frequency band is enhanced when the vibrating body 330 is long in the longitudinal direction, and therefore, this is advantageous.

In the bone conduction device 310 disclosed in FIG. 8, the center of mass 342 is located outside the first normal region 351. Accordingly, vibrations of the first weight 341 relative to the vibrating body 330 occur, so that the bone conduction device 310 achieves, with a low power consumption, a sufficient output of a sound in a frequency band corresponding to audio, based on the same principle as that of the bone conduction device 10 disclosed in FIGS. 1 to 4.

FIG. 9 is a front cross-sectional view of a bone conduction device according to Variation 4 of Embodiment 1 of the present disclosure.

A bone conduction device 410, a case 420, a body 421, a projection 422, a vibrating body 430, a support portion 431, a first holding portion 432, a second holding portion 433, a first weight 441, a center of mass 442, a second weight 446, a center of mass 447, a first normal region 451, and a second normal region 452 that are disclosed in FIG. 9 respectively correspond to the bone conduction device 10, the case 20, the body 21, the projection 22, the vibrating body 30, the support portion 31, the first holding portion 32, the second holding portion 33, the first weight 41, the center of mass 42, the second weight 46, the center of mass 47, the first normal region 51, and the second normal region 52 that are disclosed in FIGS. 1 to 4. The bone conduction device 410 disclosed in FIG. 9 differs from the bone conduction device 10 disclosed in FIGS. 1 to 4 in that the center of mass 447 of the second weight 446 is located inside the second normal region 452.

Although only the center of mass of one of the two weights is located outside the normal region in the bone conduction device 410 disclosed in FIG. 9, the center of mass 442 is located outside the first normal region 451. Accordingly, vibrations of the first weight 441 relative to the vibrating body 430 occur, so that the bone conduction device 410 achieves, with a low power consumption, a sufficient output of a sound in a frequency band corresponding to audio, based on the same principle as that of the bone conduction device 10 disclosed in FIGS. 1 to 4.

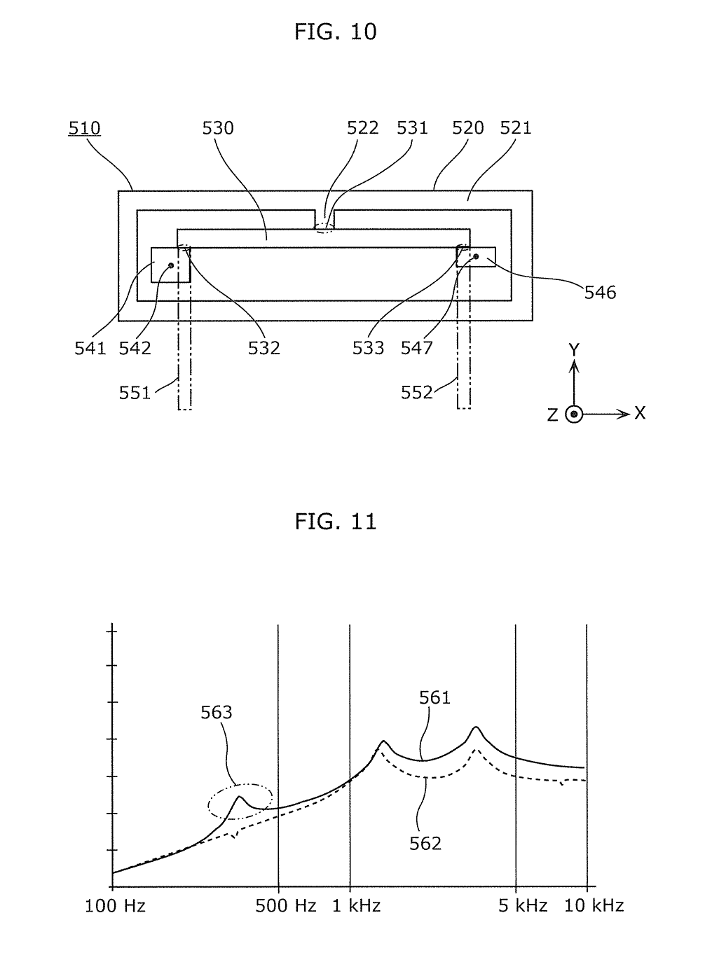

FIG. 10 is a front cross-sectional view of a bone conduction device according to Variation 5 of Embodiment 1 of the present disclosure. A bone conduction device 510, a case 520, a body 521, a projection 522, a vibrating body 530, a support portion 531, a first holding portion 532, a second holding portion 533, a first weight 541, a center of mass 542, a second weight 546, a center of mass 547, a first normal region 551, and a second normal region 552 that are disclosed in FIG. 10 respectively correspond to the bone conduction device 10, the case 20, the body 21, the projection 22, the vibrating body 30, the support portion 31, the first holding portion 32, the second holding portion 33, the first weight 41, the center of mass 42, the second weight 46, the center of mass 47, the first normal region 51, and the second normal region 52 that are disclosed in FIGS. 1 to 4. The bone conduction device 510 disclosed in FIG. 10 differs from the bone conduction device 10 disclosed in FIGS. 1 to 4 in that whereas the first weight 41 and the second weight 46 have the same mass, the first weight 541 and the second weight 546 have different masses.

In the bone conduction device 510 disclosed in FIG. 10, the center of mass 542 of the first weight 541 is located outside the first normal region 551, and the center of mass 547 of the second weight 546 is located outside the second normal region 552. Accordingly, vibrations of the first weight 541 and vibrations of the second weight 546 relative to the vibrating body 530 occur, so that the bone conduction device 510 achieves, with a low power consumption, a sufficient output of a sound in a frequency band corresponding to audio, based on the same principle as that of the bone conduction device 10 disclosed in FIGS. 1 to 4.

FIG. 11 is a graph showing vibration characteristics of the bone conduction device according to Variation 5 of Embodiment 1 of the present disclosure and a comparative example. FIG. 11 shows the output from the vibrating body in the same manner as in FIG. 5, and the axes shown in the drawing are the same as those in FIG. 5. Vibration characteristics 561 according to the present disclosure are the vibration characteristics of the bone conduction device 510 disclosed in FIG. 10, and vibration characteristics 562 according to the comparative example are the vibration characteristics of the bone conduction device 10 disclosed in FIGS. 1 to 4. The vibration characteristics 561 according to the present disclosure have a low-frequency peak 563 also at 1 kHz or less, and the characteristics in a low frequency band, in particular, at 1 kHz or less are enhanced. The reason seems to be that the first weight 541 and the second weight 546 have different masses. That is, a part of the vibrating body 530 that extends from the support portion 531 to the first holding portion 532 has vibration characteristics in accordance with the mass of the first weight 541, and a part thereof that extends from the support portion 531 to the second holding portion 533 has vibration characteristics in accordance with the mass of the second weight 546. Since the mass of the first weight 541 and the mass of the second weight 546 are different, these parts have different vibration characteristics, and vibrations attributed to the different vibration characteristics occur. More essentially, the cause of the enhancement seems to be that the vibration characteristics of the vibrating body 530 on the side extending from the support portion 531 to the first holding portion 532 and the vibration characteristics of the vibrating body 530 on the side extending from the support portion 531 to the second holding portion 533 are different. It seems that due to a grater number of vibration systems having different vibration characteristics than that of the bone conduction device 10 disclosed in FIGS. 1 to 4, the bone conduction device 510 has an increased number of output peaks resulting from an increased number of causes for the vibration systems to affect each other, and, furthermore, some of the output peaks have appeared in a low frequency band. In other words, it seems that the low-frequency band characteristics based on the above-described principle are achieved when the first weight 541 and the second weight 546 are not symmetric with respect to a virtual line in a vertical direction that is the thickness direction of the vibrating body 530, the virtual line extending through the support portion 531 of the vibrating body 530.

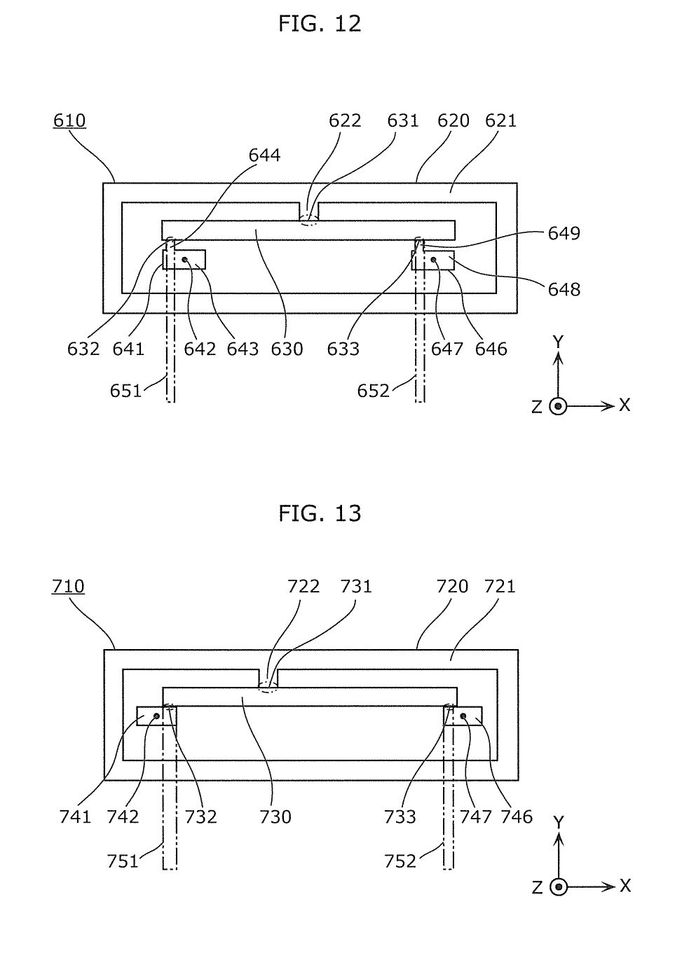

FIG. 12 is a front cross-sectional view of a bone conduction device according to Variation 6 of Embodiment 1 of the present disclosure. A bone conduction device 610, a case 620, a body 621, a projection 622, a vibrating body 630, a support portion 631, a first holding portion 632, a second holding portion 633, a first weight 641, a center of mass 642, a first weight body 643, a first connecting portion 644, a second weight 646, a center of mass 647, a second weight body 648, a second connecting portion 649, a first normal region 651, and a second normal region 652 that are disclosed in FIG. 12 respectively correspond to the bone conduction device 110, the case 120, the body 121, the projection 122, the vibrating body 130, the support portion 131, the first holding portion 132, the second holding portion 133, the first weight 141, the center of mass 142, the first weight body 143, the first connecting portion 144, the second weight 146, the center of mass 147, the second weight body 148, the second connecting portion 149, the first normal region 151, and the second normal region 152 that are disclosed in FIG. 6. The orientation of the first weight 641 and the fixing position of the second weight 646 of the bone conduction device 610 disclosed in FIG. 12 are different from the orientation of the first weight 141 and the fixing position of the second weight 146 that are disclosed in FIG. 6.

In the bone conduction device 610 disclosed in FIG. 12, the center of mass 642 of the first weight 641 is located outside the first normal region 651, and the center of mass 647 of the second weight 646 is also located outside the second normal region 652. Accordingly, vibrations of the first weight 641 and vibrations of the second weight 646 relative to the vibrating body 630 occur, so that the bone conduction device 610 achieves, with a low power consumption, a sufficient output of a sound in a frequency band corresponding to audio, based on the same principle as that of the bone conduction device 10 disclosed in FIGS. 1 to 4. Furthermore, the low-frequency band characteristics are enhanced because the distance from the support portion 631 to the first holding portion 632 is different from the distance from the support portion 631 to the second holding portion 633, or in other words, the first weight 641 and the second weight 646 are not symmetric with respect to a virtual line in a vertical direction that is the thickness direction of the vibrating body 630, the virtual line extending through the support portion 631 of the vibrating body 630.

Since the same weight is used for the first weight 641 and the second weight 646 of the bone conduction device 610 disclosed in FIG. 12, it is possible to provide an advantage that components can be used in common in the bone conduction device 610.

Although the distance from the support portion 631 to the center of mass 642 of the first weight 641 is equal to the distance from the support portion 631 to the center of mass 647 of the second weight 646 in the bone conduction device 610 disclosed in FIG. 12, they may be different distances.

FIG. 13 is a front cross-sectional view of a bone conduction device according to Variation 7 of Embodiment 1 of the present disclosure. A bone conduction device 710, a case 720, a body 721, a projection 722, a vibrating body 730, a support portion 731, a first holding portion 732, a second holding portion 733, a first weight 741, a center of mass 742, a second weight 746, a center of mass 747, a first normal region 751, and a second normal region 752 that are disclosed in FIG. 13 respectively correspond to the bone conduction device 10, the case 20, the body 21, the projection 22, the vibrating body 30, the support portion 31, the first holding portion 32, the second holding portion 33, the first weight 41, the center of mass 42, the second weight 46, the center of mass 47, the first normal region 51, and the second normal region 52 that are disclosed in FIGS. 1 to 4.

In the bone conduction device 710 disclosed in FIG. 13, the support portion 731 is not located at the center, in the longitudinal direction, of the vibrating body 730, and the bone conduction device 710 differs in this respect from the bone conduction device 10 disclosed in FIGS. 1 to 4 in which the support portion 31 is located at the center, in the longitudinal direction, of the vibrating body 30.

In the bone conduction device 710 disclosed in FIG. 13, the center of mass 742 of the first weight 741 is located outside the first normal region 751, and the center of mass 747 of the second weight 746 is located outside the second normal region 752. Accordingly, vibrations of the first weight 741 and vibrations of the second weight 746 relative to the vibrating body 730 occur, so that the bone conduction device 710 achieves, with a low power consumption, a sufficient output of a sound in a frequency band corresponding to audio, based on the same principle as that of the bone conduction device 10 disclosed in FIGS. 1 to 4. Furthermore, the low-frequency band characteristics are enhanced because the distance from the support portion 731 to the first holding portion 732 is different from the distance from the support portion 731 to the second holding portion 733, or in other words, the first weight 741 and the second weight 746 are not symmetric with respect to a virtual line in a vertical direction that is the thickness direction of the vibrating body 730, the virtual line extending through the support portion 731 of the vibrating body 730.

### Embodiment 2

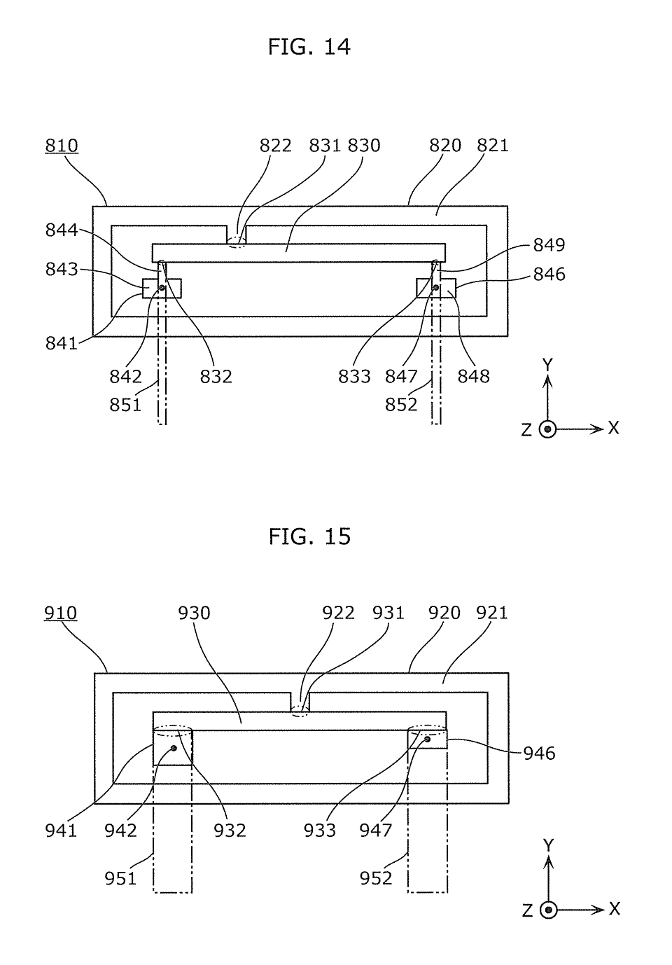

FIG. 14 is a front cross-sectional view of a bone conduction device according to Embodiment 2 of the present disclosure. A bone conduction device 810, a case 820, a body 821, a projection 822, a vibrating body 830, a support portion 831, a first holding portion 832, a second holding portion 833, a first weight 841, a center of mass 842, a first weight body 843, a first connecting portion 844, a second weight 846, a center of mass 847, a second weight body 848, a second connecting portion 849, a first normal region 851, and a second normal region 852 that are disclosed in FIG. 14 respectively correspond to the bone conduction device 610, the case 620, the body 621, the projection 622, the vibrating body 630, the support portion 631, the first holding portion 632, the second holding portion 633, the first weight 641, the center of mass 642, the first weight body 643, the first connecting portion 644, the second weight 646, the center of mass 647, the second weight body 648, the second connecting portion 649, the first normal region 651, and the second normal region 652 that are disclosed in FIG. 12.

The shapes of the first weight 841 and the second weight 846 of the bone conduction device 810 disclosed in FIG. 14 are different from the shapes of the first weight 641 and the second weight 646 that are disclosed in FIG. 12. In the bone conduction device 810, the center of mass 842 of the first weight 841 is located inside the first normal region 851, and the center of mass 847 of the second weight 846 is located inside the second normal region 852. Accordingly, in the bone conduction device 810, no or slight, if any, vibrations are caused by the positional relationship between the center of mass 842 of the first weight 841 and the first normal region 851 and the positional relationship between the center of mass 847 of the second weight 846 and the second normal region 852 as in the case of the bone conduction device 610 disclosed in FIG. 12.

In the bone conduction device 810, the low-frequency band characteristics are enhanced because the distance from the support portion 831 to the first holding portion 832 is different from the distance from the support portion 831 to the second holding portion 833, and the first weight 841 and the second weight 846 are not symmetric with respect to a virtual line in a vertical direction that is the thickness direction of the vibrating body 830, the virtual line extending through the support portion 831 of the vibrating body 830.

FIG. 15 is a front cross-sectional view of a bone conduction device according to Variation 1 of Embodiment 2 of the present disclosure. A bone conduction device 910, a case 920, a body 921, a projection 922, a vibrating body 930, a support portion 931, a first holding portion 932, a second holding portion 933, a first weight 941, a center of mass 942, a second weight 946, a center of mass 947, a first normal region 951, and a second normal region 952 that are disclosed in FIG. 15 respectively correspond to the bone conduction device 510, the case 520, the body 521, the projection 522, the vibrating body 530, the support portion 531, the first holding portion 532, the second holding portion 533, the first weight 541, the center of mass 542, the second weight 546, the center of mass 547, the first normal region 551, and the second normal region 552 that are disclosed in FIG. 10. The bone conduction device 910 disclosed in FIG. 15 differs from the bone conduction device 510 disclosed in FIG. 10 in that the center of mass 942 of the first weight 941 is located inside the first normal region 951 and the center of mass 947 of the second weight 946 is located inside the second normal region 952, and that the first weight 941 and the second weight 946 have different masses.

In the bone conduction device 910 disclosed in FIG. 15, no or slight, if any, vibrations are caused by the positional relationship between the center of mass 942 of the first weight 941 and the first normal region 951 and the positional relationship between the center of mass 947 of the second weight 946 and the second normal region 952. However, in the bone conduction device 910, the first weight 941 and the second weight 946 have different masses, so that the low-frequency band characteristics are enhanced because the first weight 941 and the second weight 946 are not symmetric with respect to a virtual line in a vertical direction that is the thickness direction of the vibrating body 930, the virtual line extending though the support portion 931 of the vibrating body 930.

### Embodiment 3

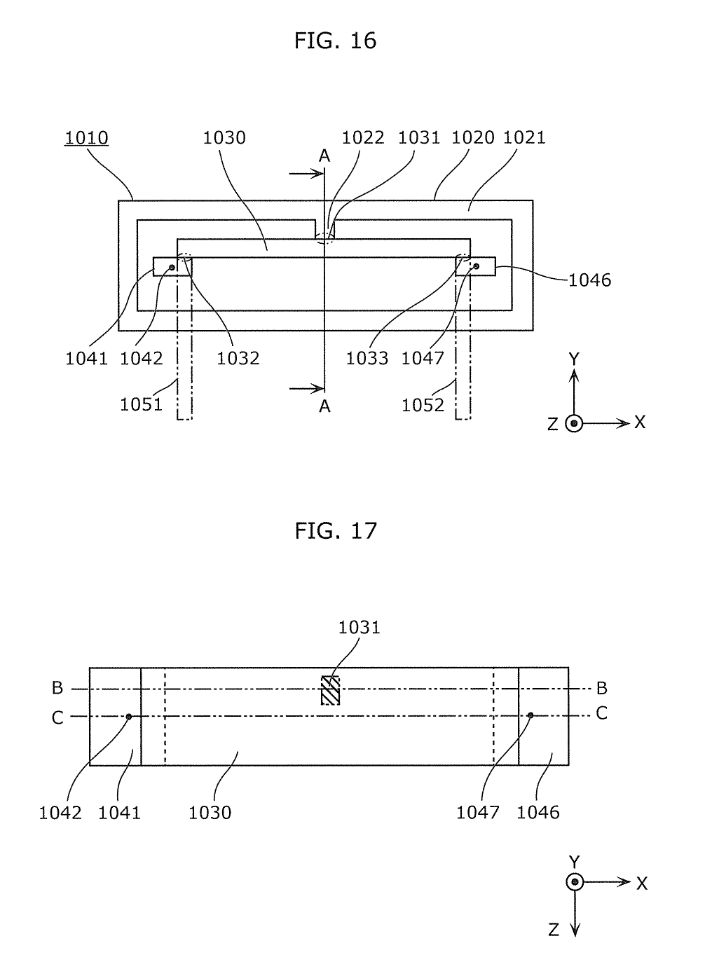

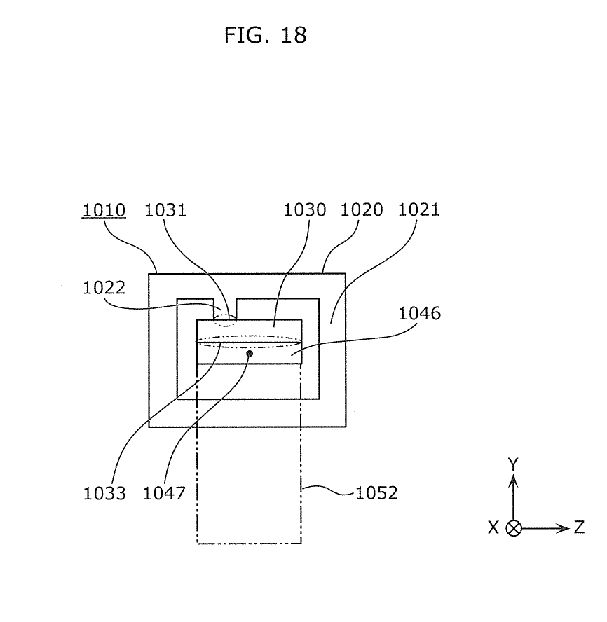

FIG. 16 is a front view of a bone conduction device according to Embodiment 3 of the present disclosure, FIG. 17 is a top view of the bone conduction device according to Embodiment 3 of the present disclosure, and FIG. 18 is a cross-sectional view taken along the line A-A in FIG. 16. The illustration of the case has been omitted from FIG. 17. In FIG. 17, the hatched region indicates a support portion 1031 instead of the cross section.

A bone conduction device 1010, a case 1020, a body 1021, a projection 1022, a vibrating body 1030, a support portion 1031, a first holding portion 1032, a second holding portion 1033, a first weight 1041, a center of mass 1042, a second weight 1046, a center of mass 1047, a first normal region 1051, and a second normal region 1052 that are disclosed in FIGS. 16 to 18 respectively correspond to the bone conduction device 10, the case 20, the body 21, the projection 22, the vibrating body 30, the support portion 31, the first holding portion 32, the second holding portion 33, the first weight 41, the center of mass 42, the second weight 46, the center of mass 47, the first normal region 51, and the second normal region 52 that are disclosed in FIGS. 1 to 4. In FIGS. 16 to 18, the X-axis is the longitudinal direction of the vibrating body 1030, the Y-axis is the thickness direction of the vibrating body 1030, and the Z-axis is the lateral direction of the vibrating body 1030. A virtual line BB in FIG. 17 extends through the central part, in the Z-axis direction, of the support portion 1031, and is parallel to the X-axis. A virtual line CC extends through the central part, in the Z-axis direction, of the vibrating body 1030, the first weight 1041, and the second weight 1046, and is parallel to the X-axis.

The bone conduction device 1010 shown in FIGS. 16 to 18 differs from the bone conduction device 10 disclosed in FIGS. 1 to 4 in that the vibrating body 1030, the first weight 1041, and the second weight 1046 are not symmetric with respect to the virtual line BB that extends through the central part, in the lateral direction, of the vibrating body 1030 of the support portion 1031 and is parallel to the longitudinal direction of the vibrating body 1030.

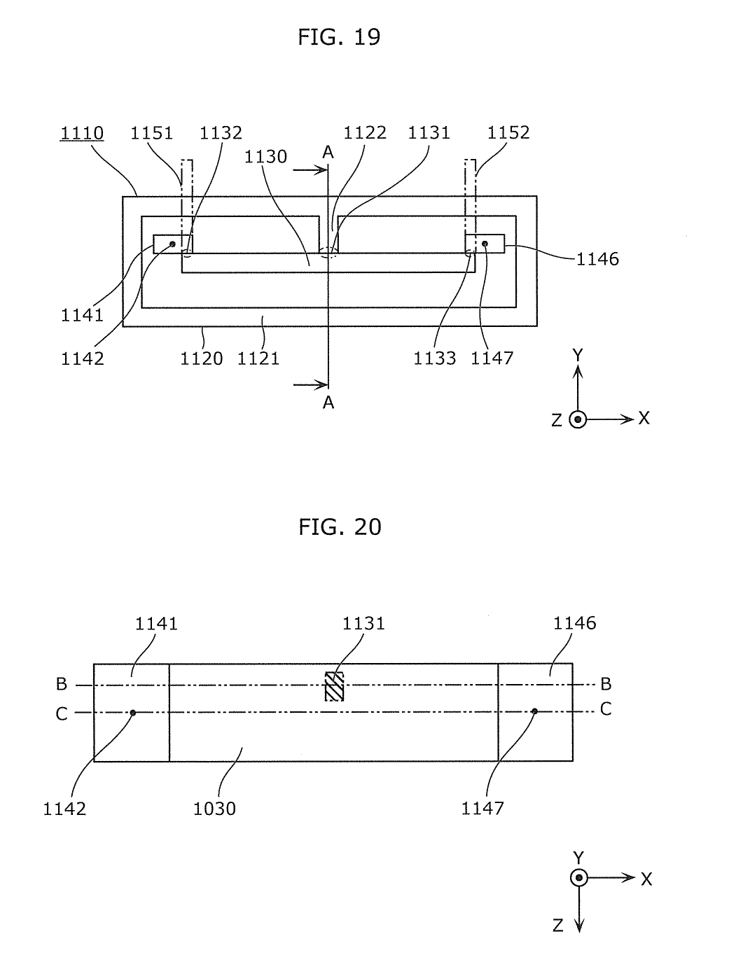

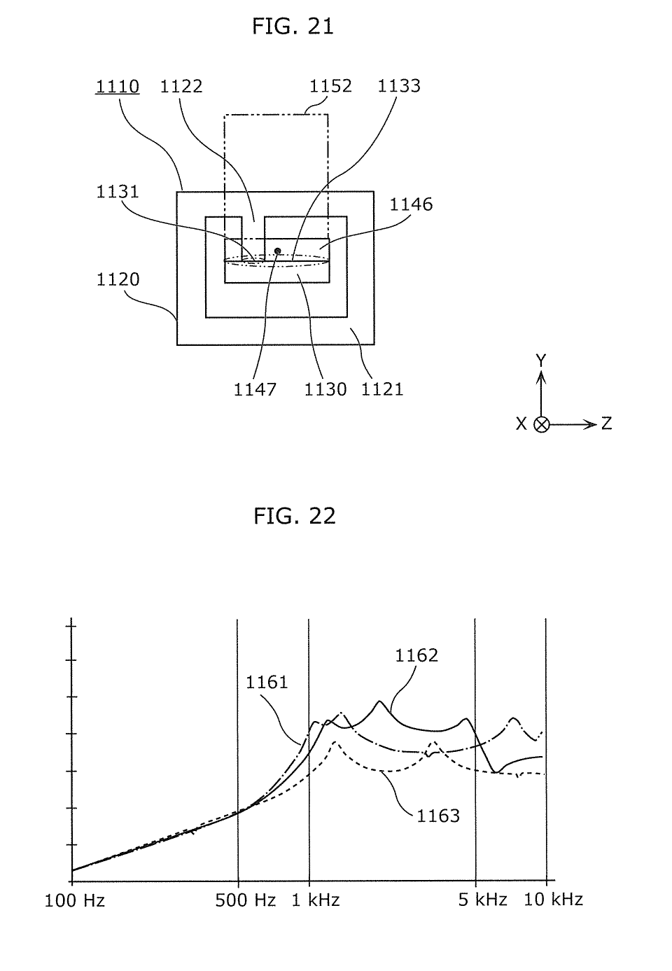

FIG. 19 is a front cross-sectional view of a bone conduction device according to Variation 1 of Embodiment 3 of the present disclosure, FIG. 20 is a top view of the bone conduction device according to Variation 1 of Embodiment 3 of the present disclosure, and FIG. 21 is a cross-sectional view taken along the line A-A in FIG. 19. The illustration of the case has been omitted from FIG. 20. In FIG. 20, the hatched region indicates a support portion 1131 instead of the cross section.

A bone conduction device 1110, a case 1120, a body 1121, a projection 1122, a vibrating body 1130, a support portion 1131, a first holding portion 1132, a second holding portion 1133, a first weight 1141, a center of mass 1142, a second weight 1146, a center of mass 1147, a first normal region 1151, and a second normal region 1152 that are described in FIGS. 19 to 21 respectively correspond to the bone conduction device 1010, the case 1020, the body 1021, the projection 1022, the vibrating body 1030, the support portion 1031, the first holding portion 1032, the second holding portion 1033, the first weight 1041, the center of mass 1042, the second weight 1046, the center of mass 1047, the first normal region 1051, and the second normal region 1052 that are disclosed in FIGS. 16 to 18.

In FIGS. 19 to 21, the X-axis is the longitudinal direction of the vibrating body 1130, the Y-axis is the thickness direction of the vibrating body 1130, and the Z-axis is the lateral direction of the vibrating body 1130. A virtual line BB in FIG. 20 extends through the central part, in the Z-axis direction, of the support portion 1131, and is parallel to the X-axis. A virtual line CC extends through the central part, in the Z-axis direction, of the vibrating body 1130, the first weight 1141, and the second weight 1146, and is parallel to the X-axis. The virtual line CC extends through the center of mass 1142 of the first weight 1141 and the center of mass 1147 of the second weight 1146 in plan view.

In the bone conduction device 1110 described in FIGS. 19 to 21, as in the bone conduction device 1010 described in FIGS. 16 to 18, the vibrating body 1130, the first weight 1141, and the second weight 1146 are not symmetric with respect to the virtual line BB that extends through the central part, in the lateral direction, of the vibrating body 1130 of the support portion 1131 and is parallel to the longitudinal direction of the vibrating body 1130.

The bone conduction device 1110 described in FIGS. 19 to 21 differs from the bone conduction device 1010 described in FIGS. 16 to 18 in the attachment positions of the first weight 1141 and the second weight 1146 to the vibrating body 1130. Specifically, in the bone conduction device 1010 described in FIGS. 16 to 18, the first weight 1041 and the second weight 1046 are attached to a surface of of the vibrating body 1030 that opposes a surface on which the support portion 1031 is provided. In contrast, in the bone conduction device 1110 described in FIGS. 19 to 21, the first weight 1141 and the second weight 1146 are attached to the same surface as the support portion 1131 of the vibrating body 1130. To describe differently, in the bone conduction device 1010 described in FIGS. 16 to 18, the first holding portion 1032 and the second holding portion 1033 are present on a surface of the vibrating body 1030 that opposes a surface on which the support portion 1031 is provided. In contrast, in the bone conduction device 1110 described in FIGS. 19 to 21, the first holding portion 1132 and the second holding portion 1133 are present on the same surface of the support portion 1131 as the vibrating body 1130.

FIG. 22 is a graph showing vibration characteristics of the bone conduction device according Embodiment 3 of the present disclosure and Variation 1 thereof, as well as a comparative example. FIG. 22 shows the output from the vibrating body in the same manner as in FIG. 5, and the axes in the drawing are the same as those shown in FIG. 5. Vibration characteristics 1161 according to Embodiment 3 are the vibration characteristics of the bone conduction device 1010 disclosed in FIGS. 16 to 18, vibration characteristics 1162 according to Variation 1 of Embodiment 3 are the vibration characteristics of the bone conduction device 1110 disclosed in FIGS. 19 to 21, and vibration characteristics 1163 according to the comparative example are the vibration characteristics of the bone conduction device 10 disclosed in FIGS. 1 to 4.

Whereas the vibration characteristics 1163 according to the comparative example have two output peaks, the vibration characteristics 1161 according to Embodiment 3 and the vibration characteristics 1162 according to Variation 1 of Embodiment 3 each have three output peaks. In the vibration characteristics 1161 according to Embodiment 3, the vibration characteristics at a frequency of about 600 Hz to about 3 kHz are enhanced as compared with those in the vibration characteristics 1163 according to the comparative example. In particular, the characteristics at a frequency near 1 kHz are significantly enhanced. In the vibration characteristics 1162 according to Variation 1 of Embodiment 3, the vibration characteristics in the range from a frequency near 600 Hz to a frequency exceeding 5 kHz are enhanced.

The reason why three peaks are present and the vibration characteristics are enhanced for the bone conduction device 1010 described in FIGS. 16 to 18 and the bone conduction device 1110 described in FIGS. 19 to 21 is presumably as follows. For the bone conduction device 1010, the vibrating body 1030, the first weight 1041, and the second weight 1046 are not symmetric with respect to the virtual line BB that extends through the central part, in the lateral direction, of the vibrating body 1030 of the support portion 1031 and is parallel to the longitudinal direction of the vibrating body 1030. For the bone conduction device 1110, the vibrating body 1130, the first weight 1141, and the second weight 1146 are not symmetric with respect to the virtual line BB that extends through central part, in the lateral direction, of the vibrating body 1130 of the support portion 1131 and is parallel to the longitudinal direction of the vibrating body 1130. Thus, vibration characteristics are enhanced by making the vibrating body, the first weight, and the second weight asymmetric in the lateral direction of vibrating body, with respect to the central part of the support portion.

The vibration characteristics 1162 according to Variation 1 of Embodiment 3 are slightly inferior at a frequency in the neighborhood of 1 kHz to those in the vibration characteristics 1161 according to Embodiment 3, but are significantly enhanced at a frequency from about 2 kHz to 5 kHz. The reason seems to be attributed to that the output peak frequency has been shifted because the support portion 1131, the first holding portion 1132, and the second holding portion 1133 are present on the same surface of the vibrating body 1130. Although the result of evaluation may vary depending on at what frequency the vibration characteristics are to be mainly enhanced, the bone conduction device 1110 described in FIGS. 19 to 21 is suitable when the characteristics at a frequency of about 2 kHz to 5 kHz are to be mainly enhanced significantly.

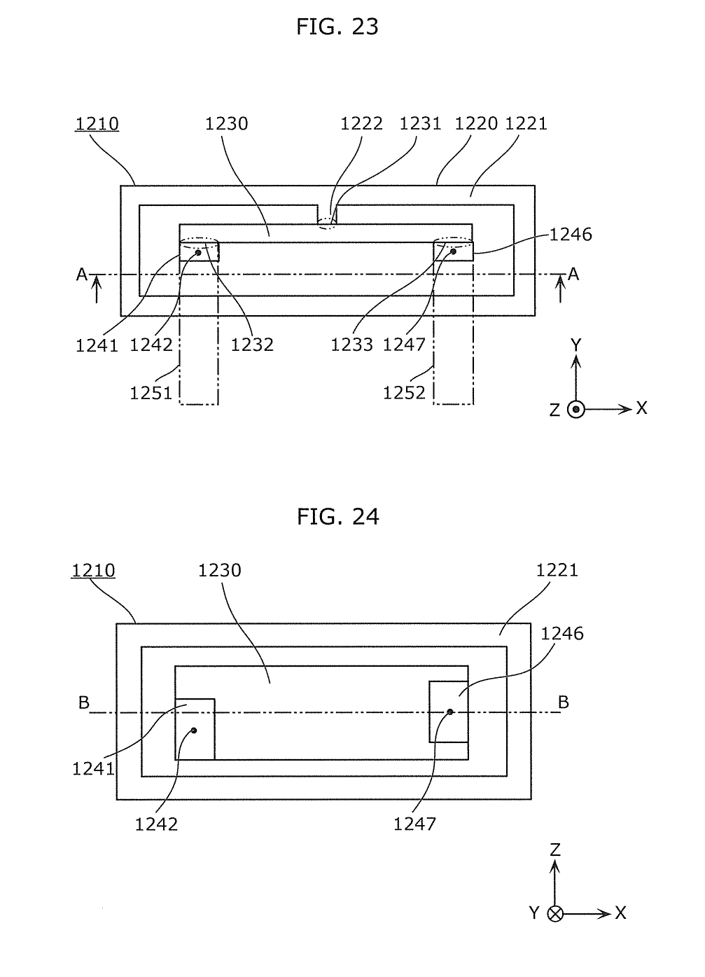

FIG. 23 is a front cross-sectional view of a bone conduction device according to Variation 2 of Embodiment 3 of the present disclosure, and FIG. 24 is a cross-sectional view taken along the line A-A in FIG. 23.

A bone conduction device 1210, a case 1220, a body 1221, a projection 1222, a vibrating body 1230, a support portion 1231, a first holding portion 1232, a second holding portion 1233, a first weight 1241, a center of mass 1242, a second weight 1246, a center of mass 1247, a first normal region 1251, and a second normal region 1252 that are disclosed in FIGS. 23 and 24 respectively correspond to the bone conduction device 910, the case 920, the body 921, the projection 922, the vibrating body 930, the support portion 931, the first holding portion 932, the second holding portion 933, the first weight 941, the center of mass 942, the second weight 946, the center of mass 947, the first normal region 951, and the second normal region 952 that are disclosed in FIG. 15. In FIGS. 23 and 24, the X-axis is the longitudinal direction of the vibrating body 1230, the Y-axis is the thickness direction of the vibrating body 1230, and the Z-axis is the lateral direction of the vibrating body 1230. A virtual line BB in FIG. 24 extends through the central part, in the Z-axis direction, of the support portion 1231, and is parallel to the X-axis. The vibrating body 1230 is symmetric relative to the virtual line BB, so that the virtual line BB extends through the center, in the lateral direction, of the vibrating body 1230.

The bone conduction device 1210 disclosed in FIGS. 23 and 24 differs from the bone conduction device 910 disclosed in FIG. 15 in that the first weight 1241 and the second weight 1246 have the same mass, and that the vibrating body 1230 and the second weight 1246 are symmetric relative to the virtual line BB, but the first weight 1241 is asymmetric relative to the virtual line BB, as shown in FIG. 24. Specifically, the first weight 1241 is located in the negative direction of the Z-axis with respect to the virtual line BB.

Since the first weight 1241 is not symmetric in the width direction of the vibrating body 1230, the low-frequency band characteristics of the bone conduction device 1210 are enhanced. Although only the first weight 1241 is made asymmetric in the width direction of the vibrating body 1230 in FIGS. 23 and 24, the second weight 1246 may also be made asymmetric.

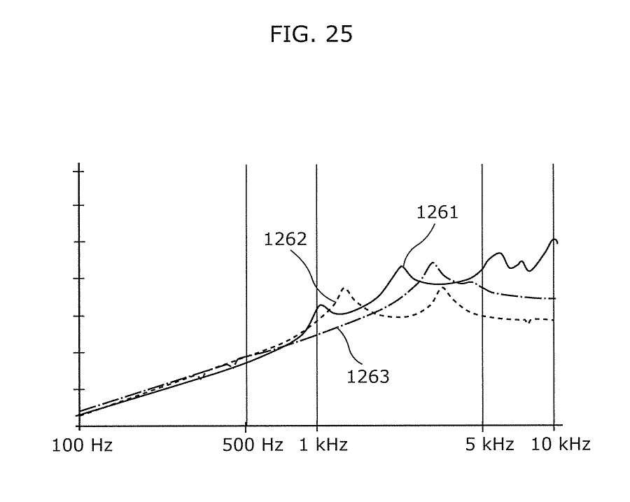

FIG. 25 is a graph showing vibration characteristics of the bone conduction device according to Variation 2 of Embodiment 3 of the present disclosure and comparative examples. FIG. 25 shows the output from the vibrating body in the same manner as in FIG. 5, and the axes in the drawing are the same as those shown in FIG. 5. Vibration characteristics 1261 according to Variation 2 of Embodiment 3 represent the vibration characteristics of the bone conduction device 1210 disclosed in FIGS. 23 and 24. Vibration characteristics 1262 according to Comparative Example 1 represent the vibration characteristics of the bone conduction device 10 shown in FIGS. 1 to 4, or in other words, the vibration characteristics 61 according to the present disclosure shown in FIG. 5. Vibration characteristics 1263 according to Comparative Example 2 represent the vibration characteristics 62 of the comparative example shown in FIG. 5.

As shown in FIG. 25, the vibration characteristics 1261 according to Variation 2 of Embodiment 3 have output peaks at a frequency near 1 kHz and a frequency near 1.2 kHz, and the output characteristics in this frequency band are enhanced as compared with those of the vibration characteristics 1263 according to Comparative Example 2. Although the vibration characteristics 1261 according to Variation 2 of Embodiment 3 are reduced in the neighborhood of 1.05 kHz as compared with those of the vibration characteristics 1262 according to Comparative Example 1, the vibration characteristics 1261 are generally enhanced at the overall frequencies.

Note that the bone conduction devices disclosed in Embodiments 1, 2, and 3 are merely examples, and the scope of the present disclosure is not limited thereto.

For example, although the projections 22, 122, 222, 322, 422, 522, 622, 722, 822, 922, 1022, 1122, and 1222 are part of the cases 20, 120, 220, 320, 420, 520, 620, 720, 820, 920, 1020, 1120, and 1220, respectively, they may be part of the vibrating bodies 30, 130, 230, 330, 430, 530, 630, 730, 830, 930, 1030, 1130, and 1230, respectively, or may each be an independent object called a piston. A stainless steel material may be used for the piston.

Although the first weights 141, 241, 641, and 841 include the first connecting portions 144, 244, 644, and 844 that are integrated with the first weight bodies 143, 243, 643, and 843, respectively, they may constitute a combination of separate objects, rather than being integrated. Likewise, although the second weights 146, 246, 646, and 846 include the second connecting portions 149, 249, 649, and 849 that are integrated with the second weight bodies 148, 248, 648, and 848, respectively, they may constitute a combination of separate objects, rather than being integrated.

In FIGS. 1 to 4, 6, 7, and 16 to 21, the first weight and the second weight are each symmetric with respect to a virtual line in a direction perpendicular to the vibrating body that is the thickness direction of the vibrating body, the virtual line extending through the support portion of the vibrating body. However, they may be each made asymmetric. As the method for making the first weight and the second weight asymmetric, it is possible to use a method in which the mass of the first weight is made different from the mass of the second weight, a method in which the distance from the support portion to the first holding portion is made different from the distance from the support portion to the second holding portion, a method in which the position of the first holding portion is made different from the position of the second holding portion in the lateral direction of the vibrating body, or a plurality of these methods.

In FIGS. 9, 10, 12, 13, 14, 15, 23, and 24, the first weight and the second weight are each already asymmetric with respect to a virtual line in a direction perpendicular to the vibrating body that is the thickness direction of the vibrating body, the virtual line extending through the support portion of the vibrating body. However, it is possible to additionally use another method for making them asymmetric.

In FIGS. 1 to 4, 6 to 10, and 12 to 15, the vibrating body and the first weight, or the vibrating body, the first weight and the second weight, if the second weight is also included, are each symmetric relative to the virtual line along the longitudinal direction of the vibrating body, the virtual line extending through the center, in the lateral direction, of the support portion of the vibrating body. However they may be made asymmetric.

In all of the drawings, the vibrating body is an object in which layers of a piezoelectric material are stacked. However, the vibrating body may be a single piece of a piezoelectric material. Alternatively, the vibrating body may have a structure in which a piezoelectric material is attached to a beam. As the piezoelectric material, it is possible to use a ceramic piezoelectric material, a MEMS (Micro Electro Mechanical Systems) piezoelectric material, or the like. The method for driving the vibrating body is not limited to a piezoelectric method, and may be an electrostatic method, or an electromagnetic method using a coil or a magnet.

As described above, the bone conduction device according to any of the embodiments and the variations of the embodiments of the present disclosure enhances the vibration characteristics by including a plurality of vibration systems, thus reducing the power consumption or increasing the output.

Although only some exemplary embodiments of the present disclosure have been described in detail above, those skilled in the art will readily appreciate that many modifications are possible in the exemplary embodiments without materially departing from the novel teachings and advantages of the present disclosure. Accordingly, all such modifications are intended to be included within the scope of the present disclosure.

### INDUSTRIAL APPLICABILITY

The present disclosure can be used for a bone conduction device using a bone-conducted sound, and is useful.

1. A bone conduction device comprising:
a case that transmits bone-conducted vibrations;
a vibrating body disposed in the case; and
a first weight held by the vibrating body, wherein
the vibrating body includes a support portion supported by the case and a first holding portion that holds the first weight, and
a center of mass of the first weight is located outside a first normal region of the vibrating body in the first holding portion.

2. The bone conduction device according to claim 1, further comprising a second weight, wherein
the vibrating body further includes, on a side opposite to the first holding portion across the support portion, a second holding portion that holds the second weight, and
a center of mass of the second weight is located outside a second normal region of the vibrating body in the second holding portion.

3. The bone conduction device according to claim 2, wherein
the vibrating body has a plate shape having a longitudinal direction, a lateral direction, and a thickness direction, and
the first weight and the second weight are asymmetric relative to a virtual line in the thickness direction of the vibrating body, the virtual line extending through the support portion.

4. The bone conduction device according to claim 3, wherein
the first weight and the second weight have different masses.

5. The bone conduction device according to claim 1, wherein
the vibrating body has a plate shape having a longitudinal direction, a lateral direction, and a thickness direction,
the vibrating body includes a first end portion in the longitudinal direction, and a second end portion located on a side opposite to the first end portion,
the support portion is located at the first end portion of the vibrating body, and
the first holding portion is located at the second end portion of the vibrating body.

6. The bone conduction device according to claim 1, wherein
the vibrating body has a plate shape having a longitudinal direction, a lateral direction, and a thickness direction, and
the vibrating body and the first weight are asymmetric relative to a virtual line along the longitudinal direction of the vibrating body, the virtual line extending through a central part of the support portion.

7. The bone conduction device according to claim 3, wherein
the vibrating body, the first weight, and the second weight are asymmetric relative to a virtual line along the longitudinal direction of the vibrating body, the virtual line extending through a central part of the support portion.

8. The bone conduction device according to claim 6, wherein
the first weight and the second weight have different masses.

9. A bone conduction device comprising:
a case;
a vibrating body disposed in the case and having a plate shape having a longitudinal direction, a lateral direction, and a thickness direction; and
a first weight and a second weight that are held by the vibrating body, wherein
the vibrating body includes a support portion supported by the case, and
the first weight and the second weight are asymmetric relative to a virtual line in the thickness direction of the vibrating body, the virtual line extending through the support portion.

10. The bone conduction device according to claim 9, wherein
the vibrating body, the first weight, and the second weight are asymmetric relative to a virtual line along the longitudinal direction of the vibrating body, the virtual line extending through a central part of the support portion.

11. The bone conduction device according to claim 9, wherein
the second weight is asymmetric relative to a virtual line along the longitudinal direction of the vibrating body, the virtual line extending through a central part of the support portion.

12. A bone conduction device comprising:
a case;
a vibrating body disposed in the case and having a plate shape having a longitudinal direction, a lateral direction, and a thickness direction; and
a first weight held by the vibrating body,
the vibrating body including a support portion supported by the case, and
the vibrating body and the first weight being asymmetric relative to a virtual line along the longitudinal direction of the vibrating body, the virtual line extending through a central part of the support portion.

13. The bone conduction device according to claim 1, wherein
a flexural modulus of the case is 1/10 of a flexural modulus of the vibrating body.

14. The bone conduction device according to claim 9, wherein
a flexural modulus of the case is 1/10 of a flexural modulus of the vibrating body.

15. The bone conduction device according to claim 12, wherein
a flexural modulus of the case is 1/10 of a flexural modulus of the vibrating body.
